(19) United States
(12) Patent Application Publication
(10) Pub. No.: US 2025/0076277 A1
(43) Pub. Date: Mar. 6, 2025

(54) METHOD OF EVALUATING RESIN MATERIAL, RESIN MATERIAL, METHOD OF PRODUCING RESIN MATERIAL, ELECTRONIC COMPONENT DEVICE, AND METHOD OF PRODUCING ELECTRONIC COMPONENT DEVICE

(71) Applicant: RESONAC CORPORATION, Minato-ku, Tokyo (JP)

(72) Inventors: Takeshi SAITO, Chiyoda-ku, Tokyo (JP); Kazuya KAMIMURA, Chiyoda-ku, Tokyo (JP)

(21) Appl. No.: 18/721,865

(22) PCT Filed: Dec. 23, 2021
(86) PCT No.: PCT/JP2021/048025

§ 371 Date: Jun. 20, 2024

(51) Int. Cl. G01N 33/44

(52) U.S. Cl. CPC G01N 33/442

(57) ABSTRACT

A method of evaluating a resin material, the method comprising: preparing a test piece comprising a resin material and a metal film disposed at a surface of the resin material; subjecting the test piece to pretreatment (1) and pretreatment (2), in order, wherein pretreatment (1) is maintaining the test piece in an environment of from 60° C. to 100° C. and a relative humidity of from 60% to 100% for at least 15 hours, and pretreatment (2) is heating the test piece under a condition with a maximum temperature of at least 200° C.; and conducting a peeling test for the metal film of the test piece after the pretreatments.

### TECHNICAL FIELD

The present disclosure relates to a method of evaluating a resin material, a resin material, a method of producing a resin material, an electronic component device, and a method of producing an electronic component device.

### BACKGROUND ART

Resin materials including a thermosetting resin, such as epoxy resin, are widely used as a sealant that protects a circumference of electronic components in electronic component devices, such as semiconductor packages (for example, Patent Document 1).

### PRIOR ART DOCUMENT

### Patent Document

Patent Document 1: Japanese Application Laid-Open No. 2021-027315

### SUMMARY OF THE INVENTION

### Problem to be Solved by the Invention

The packaging density of electronic components in electronic component devices tends to increase as the electronic component devices of recent times become smaller in size and higher in functions. As a result, there is a problem in that electromagnetic wave interference, which occurs between electronic component devices and electronic devices around the same, may be caused by noises generated from the electronic component devices. In order to suppress the occurrence of electromagnetic wave interference, a metal film is disposed around the electronic component devices.

A metal film is typically disposed at a surface of a sealant that protects a circumference of electronic components. In order to suppress the occurrence of electromagnetic wave interference effectively, the metal film preferably has a high degree of adhesion with respect to a sealant. However, a method of evaluating the adhesion of a metal film with respect to a sealant in an appropriate manner has yet to be established.

In view of the foregoing, an embodiment of the present disclosure aims to provide a method of evaluating a resin material that achieves appropriate evaluation of adhesion of a metal film. A further embodiment of the present disclosure aims to provide a resin material to which a metal film exhibits excellent adhesion, and a method of producing the resin material. A further embodiment of the present disclosure aims to provide an electronic component device in which occurrence of electromagnetic wave interference is suppressed, and a method of producing the electronic component device.

### Means for Solving the Problem

The means for solving the aforementioned problem include the following embodiments.
<1>A method of evaluating a resin material, the method comprising:
   preparing a test piece comprising a resin material and a metal film disposed at a surface of the resin material;
   subjecting the test piece to pretreatment (1) and pretreatment (2), in order, wherein pretreatment (1) is maintaining the test piece in an environment of from 60° C. to 100° C. and a relative humidity of from 60% to 100° C. for at least 15 hours, and pretreatment (2) is heating the test piece under a condition with a maximum temperature of at least 200° C.; and
   conducting a peeling test for the metal film of the test piece after the pretreatments.
<2>The method of evaluating a resin material according to <1>, wherein the peeling test is a crosscut test.
<3>The method of evaluating a resin material according to <1>or <2>, wherein the resin material is a sealant for an electronic component device.
<4>The method of evaluating a resin material according to any one of <1>to <3>, wherein the resin material comprises an epoxy resin.
<5>A resin material, from which a metal film is not peeled off when a peeling test is conducted by a crosscut test in the method of evaluating a resin material according to any one of <1>to <4>.
<6>A method of producing a resin material, the method comprising selecting a raw material based on information obtained from the method of evaluating a resin material according to any one of <1>to <4>.
<7>An electronic component device, comprising a support, an element disposed on the support, a resin material disposed around the element, and a metal film disposed around the resin material, wherein:
   the resin material is a resin material from which a metal film is not peeled off when a peeling test is conducted by a crosscut test in the method of evaluating a resin material according to any one of <1>to <4>.
<8>A method of producing an electronic component device, the electronic component device comprising a support, an element disposed on the support, a resin material disposed around the element, and a metal film disposed around the resin material, and
   the method comprising selecting the resin material based on information obtained from the method of evaluating a resin material according to any one of <1>to <4>.

### EFFECT OF THE INVENTION

According to an embodiment of the present disclosure, a method of evaluating a resin material that achieves appropriate evaluation of adhesion of a metal film is provided. According to a further embodiment of the present disclosure, a resin material to which a metal film exhibits excellent adhesion, and a method of producing the resin material are provided. According to a further embodiment of the present disclosure, an electronic component device in which occurrence of electromagnetic wave interference is suppressed, and a method of producing the electronic component device are provided.

### EMBODIMENTS FOR IMPLEMENTING THE INVENTION

In the present disclosure, the definition of the term “step” includes not only an independent step which is distinguishable from another step, but also a step which is not clearly distinguishable from another step, as long as the purpose of the step is achieved.

In the present disclosure, any numerical range described using the expression “from * to” represents a range in which numerical values described before and after the “to” are included in the range as a minimum value and a maximum value, respectively.

In a numerical range described in stages, in the present disclosure, an upper limit value or a lower limit value described in one numerical range may be replaced with an upper limit value or a lower limit value in another numerical range described in stages. Further, in a numerical range described in the present disclosure, the upper limit value or the lower limit value in the numerical range may be replaced with a value shown in the Examples.

In the present disclosure, each component may include plural kinds of substances corresponding to the component. In a case in which plural kinds of substances corresponding to each component are present in a composition, the content ratio or content of each component refers to the total content ratio or content of the plural kinds of substances present in the composition, unless otherwise specified.

In the present disclosure, particles corresponding to each component may include plural kinds of particles. In a case in which plural kinds of particles corresponding to each component are present in a composition, the particle size of each component refers to the value of the particle size of a mixture of the plural kinds of particles present in the composition, unless otherwise specified.

### <Method of Evaluating Resin Material>

The method of evaluating a resin material according to the present disclosure includes:
preparing a test piece comprising a resin material and a metal film disposed at a surface of the resin material;
subjecting the test piece to pretreatment (1) and pretreatment (2), in order, wherein pretreatment (1) is maintaining the test piece in an environment of from 60° C. to 100° C. and a relative humidity of from 60% to 100° C. for at least 15 hours, and pretreatment (2) is heating the test piece under a condition with a maximum temperature of at least 200° C.; and
conducting a peeling test for the metal film of the test piece after the pretreatments.

According to the aforementioned method, it is possible to evaluate the adhesion of a metal film with respect to a resin material in an appropriate manner. For example, when a resin material is used as a sealing material for electronic component devices, it is possible to evaluate the adhesion of a metal film that is disposed around the sealing material in an appropriate manner.

### (Preparation of Test Piece)

In the preparation of a test piece, the method for disposing a metal film at a surface of a resin material is not particularly limited, and examples thereof include sputtering, evaporation, plating and paste application.

The material for the metal film is not particularly limited, and examples thereof include copper, silver, iron, nickel, aluminum, titanium, vanadium, chromium, and alloys including these metals.

The thickness of the metal film is not particularly limited. For example, the thickness of the metal film may be selected from 10 nm to 10000 nm.

### (Pretreatment)

In the pretreatment, pretreatment (1) and pretreatment (2) are performed in this order.
(1) maintaining the test piece in an environment of from 60° C. to 100° C. and a relative humidity of from 60% to 100° C. for at least 15 hours (hereinafter, also referred to as a hygroscopic treatment)
(2) heating the test piece under a condition with a maximum temperature of at least 200° C. (hereinafter, also referred to as a thermal treatment)

In the hygroscopic treatment, the test piece is maintained in an environment of from 60° C. to 100° C. and a relative humidity of from 60% to 100° C. for at least 15 hours.

The temperature for the hygroscopic treatment is not particularly limited as long as it is 60° C. to 100° C., and may be 85° C., for example.

The relative humidity for the hygroscopic treatment is not particularly limited at long as it is from 60% to 100° C., and may be 85%, for example.

The time for the hygroscopic treatment is not particularly limited as long as it is 15 hours or more, and may be from 15 hours to 200 hours, or may be 24 hours, for example.

In the thermal treatment, the test piece is heated under a condition with a maximum temperature of at least 200° C.

The maximum temperature for the thermal treatment is not particularly limited as long as it is 200° C. or more, and may be from 200° C. to 300° C., or may be 260° C., for example.

The thermal treatment may be performed in the air or in an inert atmosphere such as nitrogen. When the metal film is formed of a metal that is prone to oxidation, such as copper, the thermal treatment is preferably performed in an inert atmosphere.

The number of times of performing the thermal treatment is not particularly limited. From the viewpoint of accuracy of the evaluation, the thermal treatment is preferably performed at least twice, more preferably at least three times.

### (Peel Test)

The method for performing the peel test is not particularly limited, and examples thereof include a crosscut test, a peel test and a scratch test.

The standard used for the evaluation of the results obtained in the peel test is not particularly limited, and may be determined such that a desired degree of accuracy is achieved.

### (Resin Material)

The resin material as a subject for the evaluation is not particularly limited, and any kind of resin material is applicable to the evaluation.

For example, the resin material may include a thermosetting resin such as epoxy resin, phenol resin, urea resin, melamine resin, unsaturated polyester resin, urethane resin, silicone resin and maleimide resin.

The resin material including an epoxy resin may be a cured product of a resin composition including an epoxy resin. The resin composition including an epoxy resin may include other components such as a curing agent and a curing accelerator.

### (Epoxy Resin)

Specific examples of the epoxy resin include novolac epoxy resin (such as a phenol novolac epoxy resin or an ortho-cresol novolac epoxy resin) which is obtained by epoxidizing a novolac resin obtained by condensation or co-condensation of at least one phenolic compound selected from the group consisting of phenol compounds such as phenol, cresol, xylenol, resorcinol, catechol, bisphenol A and bisphenol F, and naphthol compounds such as α-naphthol, β-naphthol and dihydroxynaphthalene, with an aliphatic aldehyde compound such as formaldehyde, acetaldehyde or propionaldehyde, in the presence of an acidic catalyst; triphenylmethane epoxy resin which is obtained by epoxidizing a triphenylmethane phenol resin obtained by condensation or co-condensation of the above-described phenolic compound with an aromatic aldehyde compound such as benzaldehyde or salicylaldehyde in the presence of an acidic catalyst; copolymerized epoxy resins which is obtained by epoxidizing a novolac resin obtained by co-condensation of any of the above-described phenol compound and the naphthol compound with an aldehyde compound in the presence of an acidic catalyst; diphenylmethane epoxy resin which is a diglycidyl ether of bisphenol A, bisphenol F or the like; biphenyl epoxy resin which is a diglycidyl ether of an alkyl-substituted or unsubstituted biphenol; stilbene epoxy resin which is a diglycidyl ether of a stilbene phenol compound; sulfur atom-containing epoxy resin which is a diglycidyl ether of bisphenol S or the like; epoxy resin which is a glycidyl ether of an alcohol such as butanediol, polyethylene glycol or polypropylene glycol; glycidyl ester epoxy resin which is a glycidyl ester of a polyvalent carboxylic acid compound such as phthalic acid, isophthalic acid or tetrahydrophthalic acid; glycidyl amine epoxy resin which is obtained by substituting an active hydrogen bound to a nitrogen atom of aniline, diaminodiphenylmethane, isocyanuric acid or the like, with a glycidyl group; dicyclopentadiene epoxy resin which is obtained by epoxidizing a co-condensed resin of dicyclopentadiene with a phenol compound; alicyclic epoxy resin which is obtained by epoxidizing an olefin bond in the molecule, such as vinylcyclohexene diepoxide, 3,4-epoxycyclohexylmethyl-3,4-epoxycyclohexane carboxylate or 2-(3,4-epoxy)cyclohexyl-5,5-spiro(3,4-epoxy)cyclohexane-m-dioxane; paraxylylene-modified epoxy resin which is a glycidyl ether of a paraxylylene-modified phenol resin; metaxylylene-modified epoxy resin which is a glycidyl ether of a metaxylylene-modified phenol resin; terpene-modified epoxy resin which is a glycidyl ether of a terpene-modified phenol resin; dicyclopentadiene-modified epoxy resin which is a glycidyl ether of a dicyclopentadiene-modified phenol resin; cyclopentadiene-modified epoxy resin which is a glycidyl ether of a cyclopentadiene-modified phenol resin; polycyclic aromatic ring-modified epoxy resin which is a glycidyl ether of a polycyclic aromatic ring-modified phenol resin; naphthalene epoxy resin which is a glycidyl ether of a naphthalene ring-containing phenol resin; halogenated phenol novolac epoxy resin; hydroquinone epoxy resin; trimethylolpropane epoxy resin; linear aliphatic epoxy resin which is obtained by oxidizing an olefin bond with a peracid such as peracetic acid; and aralkyl epoxy resin which is obtained by epoxidizing an aralkyl phenol resin such as a phenol aralkyl resin or a naphthol aralkyl resin. Epoxidized acrylic resin and the like may be referred to as the epoxy resin. The epoxy resin may be used singly, or in combination of two or more kinds thereof.

From the viewpoint of increasing the glass transition temperature of a cured product, a triphenylmethane epoxy resin, a biphenyl aralkyl epoxy resin, a naphthalene aralkyl epoxy resin and a novolac epoxy resin are preferred as an epoxy resin.

The epoxy equivalent (molecular weight/number of epoxy groups) of the epoxy resin is not particularly limited. From the viewpoint of a valance of the properties such as moldability, reflow resistance and electrical reliability, the epoxy equivalent of the epoxy resin is preferably from 100 g/eq to 1000 g/eq, more preferably from 150 g/eq to 500 g/eq.

The epoxy equivalent of the epoxy resin is measured by a method according to JIS K 7236:2009.

When the epoxy resin is solid, the softening point or the melting point of the epoxy resin is not particularly limited. From the viewpoint of moldability and reflow resistance, the softening point or the melting point of the epoxy resin is preferably from 40° C. to 180° C. From the viewpoint of handleability during the preparation of the resin composition, the softening point or the melting point of the epoxy resin is preferably from 50° C. to 130° C.

The softening point or the melting point of the epoxy resin is measured by differential scanning calorimetry (DSC) or measured by a method according to JIS K 7234:1986 (ring-and-ball method).

The content of the epoxy resin in the resin composition is preferably from 0.5% by mass to 50% by mass, more preferably from 2% by mass to 30% by mass, from the viewpoint of strength, fluidity, heat resistance and moldability.

### (Curing Agent)

The resin composition may include a curing agent. The type of the curing agent is not particularly limited, and may be selected depending on the desired properties of the resin composition. Examples of the curing agent include phenol curing agent, amine curing agent, acid anhydride curing agent, polymercaptan curing agent, polyaminoamide curing agent, isocyanate curing agent, block isocyanate curing agent, and an active ester compound. The curing agent may be used singly, or in combination of two or more kinds thereof.

Specific examples of the phenol curing agent include a polyvalent phenol compound, such as resorcin, catechol, bisphenol A, bisphenol F and a substituted or unsubstituted biphenol; a novolac phenol resin obtained by condensation or co-condensation of at least one phenollic compound selected from the group consisting of a phenol compound such as phenol, cresol, xylenol, resorcin, catechol, bisphenol A, bisphenol F, phenylphenol, and aminophenol, and a naphthol compound such as α-naphthol, β-naphthol and dihydroxynaphthalene, and an aldehyde compound such as formaldehyde, benzaldehyde or salicylaldehyde, in the presence of an acidic catalyst; an aralkyl phenol resin such as a phenol aralkyl resin, a biphenyl aralkyl phenol resin and a naphthol aralkyl resin; a p-xylylene-modified phenol resin and a m-xylylene-modified phenol resin; a melamine-modified phenol resin; a terpene-modified phenol resin; a dicyclopentadiene-modified phenol resin and a dicyclopentadiene-modified naphthol resin, which is a copolymer synthesized from the phenollic compound as described above and dicyclopentadiene; a cyclopentadiene-modified phenol resin; a polycyclic aromatic ring-modified phenol resin; a biphenyl phenol resin; a triphenylmethane phenol resin, which is obtained by condensation or co-condensation of the phenollic compound as described above with an aromatic aldehyde compound such as benzaldehyde or salicylaldehyde; and a phenol resin obtained by copolymerizing two or more of the phenol resins as described above. The phenol curing agent may be used singly or in combination of two or more kinds.

From the viewpoint of increasing the glass transition temperature of a cured product of the resin composition, a triphenylmethane phenol resin, a biphenyl aralkyl phenol resin, a naphthalene aralkyl phenol resin and a novolac phenol resin are preferred.

When the curing agent is solid, the softening point or the melting point of the curing agent is not particularly limited. From the viewpoint of moldability and reflow resistance, the softening point or the melting point of the curing agent is preferably from 40° C. to 180° C. From the viewpoint of handleability during the preparation of the resin composition, the softening point or the melting point of the curing agent is preferably from 50° C. to 130° C.

The softening point or the melting point of the curing agent is measured by the same manner as the softening point or the melting point of the epoxy resin.

The equivalent ratio of the curing agent and the epoxy resin, i.e., a ratio of the number of functional group in the curing agent with respect to the number of functional group in the epoxy resin (number of functional group in curing agent/number of functional group in epoxy resin) is not particularly limited. From the viewpoint of suppressing the amount of unreacted components, the equivalent ratio is preferably from 0.5 to 2.0, more preferably from 0.6 to 1.3. From the viewpoint of moldability and reflow resistance, the equivalent ratio is preferably from 0.8 to 1.2.

### (Curing Accelerator)

The resin composition may include a curing accelerator. The type of the curing accelerator is not particularly limited, and may be selected depending on the desired properties of the resin composition, or the like.

The amount of the curing accelerator in the resin composition is preferably from 0.1 parts by mass to 30 parts by mass with respect to 100 parts by mass of the resin component (i.e., a total amount of epoxy resin and curing agent), more preferably from 1 part by mass to 15 parts by mass. When the amount of the curing accelerator is 0.1 pars by mass or more with respect to 100 parts by mass of the resin component, the resin composition tends to cure within a short time period in a favorable manner. When the amount of the curing accelerator is 30 pars by mass or less with respect to 100 parts by mass of the resin component, the rate of curing is kept from being too high and a favorable cured product tend to be obtained.

### (Filler)

The resin composition may include a filler. The type of the filler is not particularly limited. Specific examples of the filler include inorganic materials such as fused silica, crystalline silica, glass, alumina, talc, clay and mica. It is also possible to use an inorganic filler having a fire-retardant effect. Examples of the inorganic filler having a fire-retardant effect include aluminum hydroxide, magnesium hydroxide, a composite metallic hydroxide such as a composite hydroxide of magnesium and zinc, and zinc borate.

From the viewpoint of reducing the linear expansion coefficient, the filler is preferably fused silica. From the viewpoint of achieving a high thermal conductivity, the filler is preferably alumina. The filler may be used singly or in combination of two or more kinds. Example of the configuration of the filler include a powder, beads obtained by spheronization of a powder, fibers and the like.

The volume average particle size of the filler included in the resin composition is preferably 10 μm or less, more preferably from 1 μm to 8 μm, further preferably from 2 μm to 6 μm, from the viewpoint of filling properties.

The maximum particle size of the filler included in the resin composition is preferably 50 μm or less, more preferably 30 μm or less, from the viewpoint of filling properties.

In the present disclosure, the volume average particle diameter of the filler refers to a particle size (D50) at which the accumulation in volume from the side of smaller particle size reaches 50% in a volume-based particle size distribution. The volume-based particle size distribution is obtained with a laser diffraction/scattering particle size distribution analyzer (such as LA 920, Horiba, Ltd.)

When the filler is included in the resin composition, the volume average particle diameter of the filler may be measured after removing the resin component included in the resin composition by way of thermal decomposition, dissolution or the like.

The content of the filler in the resin composition is not particularly limited. From the viewpoint of fluidity and strength, the content of the filler with respect to the total resin composition is preferably from 30% by volume to 90% by volume, more preferably from 35% by volume to 80% by volume, further preferably from 50% by volume to 80% by volume. When the content of the filler is 30% by volume or more with respect to the total resin composition, properties of a cured product such as the thermal expansion coefficient, thermal conductivity and elastic modulus tend to be further improved. When the content of the filler is 90% by volume or less with respect to the resin composition, an increase in viscosity of the resin composition is suppressed, thereby further improving the fluidity and the moldability of the resin composition.

The resin composition may include various type of additives, such as a coupling agent, an ion exchanger, a mold release agent, a fire retardant, a colorant, a silicone oil, and silicon particles, as mentioned below. As necessary, the resin composition may include additives known in the art other than the following additives.

### (Coupling Agent)

When the resin composition includes an inorganic filler, a coupling agent may be included in order to improve the adhesion between the resin component and the inorganic filler. Examples of the coupling agent include silane compounds such as epoxysilane, mercaptosilane, aminosilane, ureidosilane and vinylsilane, titanium compounds, aluminum chelate compounds, and aluminum/zirconium compounds.

When the resin composition includes a coupling agent, the amount thereof with respect to 100 parts by mass of the inorganic filler is preferably from 0.05 parts by mass to 10 parts by mass, more preferably from 0.1 parts by mass to 8 parts by mass. When the amount of the coupling agent is 0.05 parts by mass or more with respect to 100 parts by mass of the inorganic filler, adhesion with respect to a lead frame tends to be further improved. When the amount of the coupling agent is 10 parts by mass or less with respect to 100 parts by mass of the inorganic filler, moldability of a package tends to be further improved.

### (Ion Exchanger)

The resin composition may include an ion exchanger. In particular, the resin components preferably includes an ion exchanger in order to improve the resistance to moisture and the resistance to high-temperature environments. The ion exchanger is not particularly limited, and may be selected from known products. Specific examples of the ion exchanger include hydrous oxides containing at least one element selected from the group consisting of magnesium, aluminum, titanium, zirconium and bismuth, and hydrotalcites. The ion exchanger may be used singly or in combination of two or more kinds. Among the ion exchangers, a hydrotalcite represented by the following Formula (A) is preferred. In Formula (A), X is a number that satisfies 0<X≤0.5, and m is a positive number.

Mg_{(1-X)}Al_{X}(OH)₂(CO₃)_{X/2}·mH₂O . . . (A)

When the resin composition includes an ion exchanger, the content thereof is not particularly limited as long as it is enough to trap ions such as halogen ions. For example, the amount of the ion exchanger with respect to 100 parts by mass of the resin component (i.e., a total amount of the epoxy resin and the curing agent) is preferably from 0.1 parts by mass to 30 parts by mass, more preferably from 1 part by mas to 10 parts by mass.

### (Mold Release Agent)

The resin composition may include a mold release agent for the purpose of achieving a favorable releasability from a mold during the molding. The mold release agent is not particularly limited, and may be selected from known products. Specific examples of the mold release agent include higher fatty acids such as carnauba wax, montanic acid and stearic acid, metal salts of higher fatty acid, ester waxes such as montanic acid ester, and polyolefin waxes such as oxidized polyethylene and non-oxidized polyethylene. The mold release agent may be used singly or in combination of two or more kinds.

When the curable resin composition includes a mold release agent, the amount thereof with respect to 100 parts by mass of the resin component (i.e., a total amount of the epoxy resin and the curing agent) is preferably from 0.01 parts by mass to 10 parts by mass, more preferably from 0.1 parts by mass to 5 parts by mass. When the amount of the mold release agent is 0.01 parts by mass or more with respect to 100 parts by mass of the resin component, a sufficient degree of releasability tends to be achieved. When the amount of the mold release agent is 10 parts by mass or less with respect to 100 parts by mass of the resin component, a more favorable adhesion tends to be achieved.

### (Fire Retardant)

The resin composition may include a fire retardant. The fire retardant is not particularly limited, and may be selected from known products. Specific examples of the fire retardant include organic or inorganic compounds including a halogen atom, an antimony atom, a nitrogen atom or a phosphorous atom, and metallic hydroxides. The fire retardant may be used singly or in combination of two or more kinds.

When the curable resin composition includes a fire retardant, the amount thereof is not particularly limited as long as it is enough to achieve a desired fire-retardant effect. For example, the amount of the fire retardant with respect to 100 parts by mass of the resin component (i.e., a total amount of the epoxy resin and the curing agent) is preferably from 1 part by mass to 30 parts by mass, more preferably from 2 parts by mass to 20 parts by mass.

### (Colorant)

The curable resin composition may include a colorant. Examples of the colorant include carbon black, organic dyes, organic pigments, titanium oxide, minium and colcothar. The amount of the colorant may be selected depending on the purposes and the like. The colorant may be used singly or in combination of two or more kinds.

### (Silicone Particles)

The resin composition may include silicone particles. The silicone particles may be those having a core-shell structure. Examples of the silicone particles having a core-shell structure include a product obtained by coating a surface of silicone rubber particles with a silicone resin (silicone-based core-shell rubber particles).

The volume-average particle diameter of the silicone particles may be from 100 nm to 10 μm.

### <Resin Material>

The resin material according to the present disclosure is a resin material, from which a metal film is not peeled off when a peeling test is conducted by a crosscut test in the aforementioned method of evaluating a resin material.

The resin material according to the present disclosure causes a metal film to exhibit excellent adhesion to the resin material. Therefore, occurrence of electromagnetic wave interference is effectively suppressed in an electronic component device in which the resin material is used as a sealing material and a metal film is disposed around the sealing material.

The type of the resin material is not particularly limited. For example, the resin material may be the aforementioned resin composition including an epoxy resin.

### <Method of Producing Resin Material>

The method of producing a resin material according to the present disclosure is a method for producing a resin material, the method including selecting a raw material based on information obtained from the aforementioned method of evaluating a resin material.

In the present method, examples of information obtained from the method of evaluating a resin material include a result of a peel test conducted (for example, existence or non-existence of peeling in a crosscut test).

The resin material produced by the method causes a metal film to exhibit excellent adhesion to the resin material. Therefore, occurrence of electromagnetic wave interference is effectively suppressed in an electronic component device in which a resin material produced by the method is used as a sealing material and a metal film is disposed around the sealing material.

The raw material for the resin material is not particularly limited. For example, the raw material may be selected from a raw material for the aforementioned resin composition including an epoxy resin.

### <Electronic Component Device>

The electronic component device according to the present disclosure is an electronic component device, including a support, an element disposed on the support, a resin material disposed around the element, and a metal film disposed around the resin material, wherein: the resin material is a resin material from which a metal film is not peeled off when a peeling test is conducted by a crosscut test in the aforementioned method of evaluating a resin material.

The resin material included in the electronic component device according to the present disclosure causes a metal film to exhibit excellent adhesion to the resin material. Therefore, occurrence of electromagnetic wave interference is effectively suppressed in the electronic component device.

The type of the support, the element and the metal film is not particularly limited, and commonly used supports, elements and metal films may be used.

Examples of the electronic component device include those having, on a support such as a lead frame, a wired tape carrier, a wiring board, a glass or a silicon wafer, an element portion that is formed by mounting an element on the support and sealing the same with the curable resin composition. Examples of the element include active elements such as a semiconductor chip, a transistor, a diode and a thyristor and passive elements such as a capacitor, a resistor or a coil.

Specific examples of the electronic component device include ordinary resin-sealed ICs such as a DIP (dual inline package), a PLCC (plastic leaded chip carrier), a QFP (quad flat package), an SOP (small outline package), an SOJ (small outline J-lead package), a TSOP (thin small outline package) and a TQFP (thin quad flat package), which have a structure in which an element is fixed on a lead frame and a terminal portion of the element, such as a bonding pad, is connected to a lead portion with wires or bumps, and sealed with the curable resin composition by transfer molding or the like; a TCP (tape carrier package), having a structure in which an element is connected to a tape carrier with bumps, and sealed with the curable resin composition; a COB (chip on board) module, a hybrid IC and a multichip module, having a structure in which an element is connected to a wiring formed on a support by wire bonding, flip-chip bonding, soldering or the like, and sealed with the curable resin composition; and a BGA (ball grid array) and a CSP (chip size package), having a structure formed by mounting an element on a support having, at a rear surface thereof, a terminal to be connected to a wiring board, connecting the element to a wiring formed on the support with bumps or wires, and sealing the same with the curable resin composition. The curable resin composition may also be suitably used for a printed circuit board.

### <Method of Producing Electronic Component Device>

The method of producing an electronic component device according to the present disclosure is a method of producing an electronic component device, the electronic component device including a support, an element disposed on the support, a resin material disposed around the element, and a metal film disposed around the resin material, and the method including selecting the resin material based on information obtained from the aforementioned method of evaluating a resin material.

In the present method, examples of information obtained from the method of evaluating a resin material include a result of a peel test conducted (for example, existence or non-existence of peeling in a crosscut test).

The resin material, included in an electronic component device produced by the method, causes a metal film to exhibit excellent adhesion to the resin material. Therefore, occurrence of electromagnetic wave interference is effectively suppressed in an electronic component device produced by the method.

The type of the support, the element and the metal film used for the production of an electronic component device is not particularly limited, and commonly used supports, elements and metal films may be used.

### EXAMPLES

In the following, the present invention is explained in further details by referring to the Examples. However, the scope of the present invention is not limited to the Examples.

### <Preparation of Test Piece>

Test pieces were prepared using commercially available sealing materials A and B including an epoxy resin, respectively.

Specifically, a molding was formed on a support using the sealing material at a molding temperature of 175° C. and a molding time of 120 seconds. Thereafter, the support was removed from the molding, and the molding was subjected to post-curing at 175° C. for 5 hours, thereby obtaining a cured product of 235 mm×65 mm×0.7 mm. A test piece was obtained by forming a copper film (thickness: approximately 150 nm) at one of the main surfaces and the side surfaces of the cured product by sputtering.

### <Peel Test (Without Pretreatment)>

The test piece was subjected to electrolytic plating to increase a thickness of the copper film up to 20 μm, and a peel strength was measured using a peel tester (EZ-SX, Shimadzu Corporation). The measurement was conducted at a peeling angle of 90°, a peeling rate of 5 mm/min, a measurement width of 10 mm and a measurement length of 20 mm.

The result of a test piece prepared using sealing material A was 0.711 kN/m and the result of a test piece prepared using sealing material B was 0.767 kN/m.

### <Crosscut Test (Without Pretreatment)>

The test piece was subjected to a crosscut test according to JIS K 5600 with a sample number N of 3.

Specifically, incisions were formed at an interval of 1 mm to form a grid pattern of 25 cells (5 cells in length×5 cells in width) at the metal film of the test piece, and a tape was attached thereto. Within 5 minutes from the attachment of the tape, the tape was peeled off without stopping at an angle of 60° within a time of from 0.5 to 1.0 second.

The number of cells at which the metal film remains after the peeling of the tape was 25 (no peeling-off) in the test piece prepared using sealing material A and the test piece prepared using sealing material B, respectively.

### <Crosscut Test (With Pretreatment)>

The test pieces were subjected to a hygroscopic treatment in which the test pieces were maintained in an environment of 85° C. and a relative humidity of 85% for 24 hours. Thereafter, the test pieces were subjected to a thermal treatment with a maximum temperature of 260° C. in a nitrogen atmosphere. The thermal treatment was performed three times.

The test pieces after being subjected to the pretreatment were subjected to a crosscut test conducted in the same manner as the aforementioned method.

The number of cells at which the metal film remains after the peeling of the tape was 25 (no peeling-off) in the test piece prepared using sealing material A, whereas the number of cells at which the metal film remains after the peeling of the tape was zero (all peeling-off) in the test piece prepared using sealing material B.

As shown in the aforementioned results, while the test pieces prepared using sealing material A or sealing material B exhibited a substantially equivalent adhesion of a metal film when the peel test was performed without the pretreatment, there was a significant difference in the adhesion of a metal film between the test pieces when the peel test was performed with the pretreatment.

1. A method of evaluating a resin material, the method comprising:
preparing a test piece comprising a resin material and a metal film disposed at a surface of the resin material;
subjecting the test piece to pretreatment (1) and pretreatment (2), in order, wherein pretreatment (1) is maintaining the test piece in an environment of from 60° C. to 100° C. and a relative humidity of from 60% to 100% for at least 15 hours, and pretreatment (2) is heating the test piece under a condition with a maximum temperature of at least 200° C.; and
conducting a peeling test for the metal film of the test piece after the pretreatments.

2. The method of evaluating a resin material according to claim 1, wherein the peeling test is a crosscut test.

3. The method of evaluating a resin material according to claim 1, wherein the resin material is a sealant for an electronic component device.

4. The method of evaluating a resin material according to claim 1, wherein the resin material comprises an epoxy resin.

5. A resin material, from which a metal film is not peeled off when a peeling test is conducted by a crosscut test in the method of evaluating a resin material according to claim 1.

6. A method of producing a resin material, the method comprising selecting a raw material based on information obtained from the method of evaluating a resin material according to claim 1.

7. An electronic component device, comprising a support, an element disposed on the support, a resin material disposed around the element, and a metal film disposed around the resin material, wherein:
the resin material is a resin material from which a metal film is not peeled off when a peeling test is conducted by a crosscut test in the method of evaluating a resin material according to claim 1.

8. A method of producing an electronic component device, the electronic component device comprising a support, an element disposed on the support, a resin material disposed around the element, and a metal film disposed around the resin material, and
the method comprising selecting the resin material based on information obtained from the method of evaluating a resin material according to claim 1.
